(19) United States
(12) Patent Application Publication
(10) Pub. No.: US 2006/0079774 A1
(43) Pub. Date: Apr. 13, 2006

(54) Microwave biopsy probe

(76) Inventor: Wendell Anderson, Santa Rosa, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/961,812

(22) Filed: Oct. 8, 2004

(51) Int. Cl. A61B 8/00

(52) U.S. Cl. 600/442; 600/562

(57) ABSTRACT

A biopsy probe includes an impedance probe slidably disposed within a biopsy needle.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

An excisional biopsy is a medical procedure where a sample of tissue is removed from a patient for examination. Biopsies are important in determining whether a lesion (“lump”) in tissue, such as breast tissue, is malignant or benign. Biopsies are also often performed on lesions that have been completely removed (“complete excisional biopsy”) from the patient. However, lesions are often benign. Removal of all lesions would subject the patient to unnecessary surgical procedures and the resultant discomfort, healing time, and scarring.

Other excisional biopsy techniques remove a small portion of a lesion for analysis. If the lesion is determined to be benign, complete excision is usually not required. If the lesion is malignant, then a complete excision of the lesion is usually performed. Various types of devices and procedures have been developed to permit removal of a small amount of tissue or fluid from a tissue site for medical examination. Several biopsy techniques using biopsy needles, such as fine needle aspiration, core needle biopsy, vacuum-assisted biopsy, and large-core biopsy, have been developed. Generally, performing a biopsy using a biopsy needle can be done with local anesthesia, rather than under general anesthesia, which is sometimes used in a complete excisional biopsy. Needle biopsy also can be done in much less time, does not require an operating room, removes much less tissue, is less invasive, and healing time is shorter.

Biopsy needles are guided to the desired tissue site using stereoscopic x-ray imaging, ultrasound imaging, or other imaging techniques or combination of techniques. The surgeon watches the progression of the biopsy needle on an electronic display screen as the needle is inserted into the patient. Some biopsy needles have specially shaped tips or other features to enhance their ultrasonic “signature.” Once the biopsy needle is inserted into the tissue site being evaluated, some of the tissue and/or surrounding fluid is removed and evaluated. Another technique guides a fine, localizing needle to the tissue site through a cannula, which is a tube with a cutting device, such as a sharpened, serrated end. The cannula is guided over and along the localizing needle to the lesion, where the cannula cuts out a sample of tissue for analysis. However, tissue samples are sent for analysis when using excisional biopsy techniques, whether complete or needle biopsy.

Receiving the results of the tissue analysis can take several hours to several days. It is desirable that a physician be able to determine whether further analysis or removal of a lesion is necessary without having to wait for the results of analysis of removed tissue.

### BRIEF SUMMARY OF THE INVENTION

A biopsy probe includes an impedance probe slidably disposed within a biopsy needle.

### BRIEF DESCRIPTION OF THE DRAWINGS

FIG. 1A is an isometric view of an impedance probe according to an embodiment of the invention.

FIG. 1B is a cross section of the impedance probe of FIG. 1A

FIG. 2A is a simplified cross section of biopsy probe according to an embodiment of the present invention.

FIG. 2B shows the biopsy probe of FIG. 2A with the biopsy needle retracted to expose the end of the impedance probe.

FIG. 3A is an isometric view of an automated biopsy impedance probe.

FIG. 3B is an isometric view of the automated probe with the carriage rotated down to allow removal of the biopsy needle.

FIG. 3C is an isometric view of a portion of the automated probe of FIGS. 3A and 3B.

### DETAILED DESCRIPTION OF THE EMBODIMENTS

I. Introduction

The present invention enables analysis of lesions that is less invasive than excision biopsy techniques and provides analytical results of the biopsy much quicker than conventional biopsy techniques relying on analysis of removed tissue or fluids. An impedance probe is used to evaluate tissue in and around a region. In some embodiments, the impedance probe operates at microwave frequencies and is relatively small. A biopsy probe is used to cut a path through tissue to the desired evaluation site, and the impedance probe is extended through the end of the biopsy needle to measure the electrical impedance of the evaluation site. The biopsy needle protects the impedance probe during insertion.

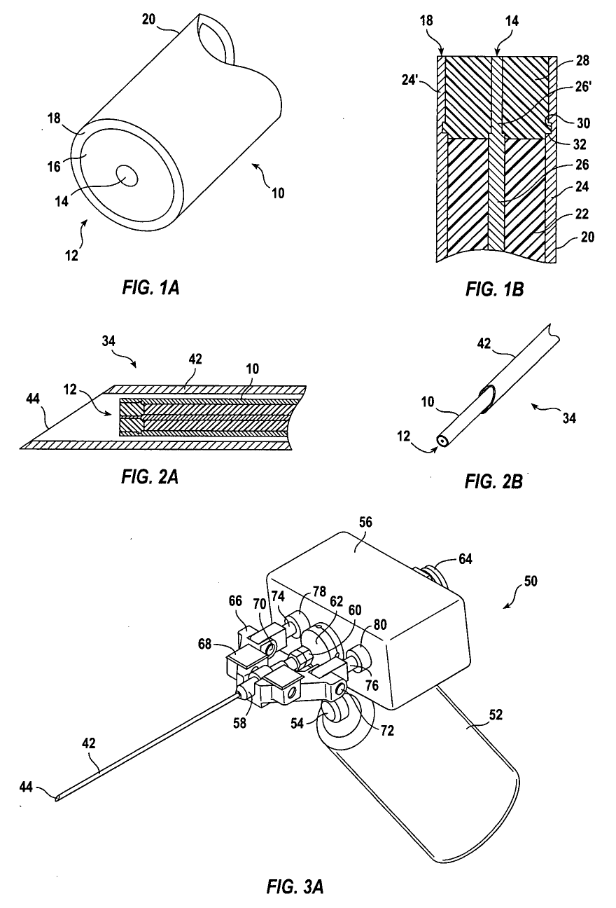

FIG. 1A is an isometric view of an impedance probe 10 according to an embodiment of the invention. The impedance probe has a sealed tip 12 having a center conductor end 14, a dielectric end 16, and an outer conductor end 18, forming an “open-ended” coaxial probe. The dielectric end 16 is a dielectric sealant, such as an epoxy encapsulant or other thermosetting or thermoplastic encapsulant or sealant. In a particular embodiment, the dielectric end is STYCAST® E-1050™ epoxy encapsulant, available from EMERSON & CUMING of Billerica, Md. The body 20 of the impedance probe is a section of coaxial transmission line, such as rigid or semi-rigid coaxial transmission line. In a particular embodiment, the coaxial transmission line has an outside diameter of not greater than about 0.79 mm (0.031 inches, commonly called “031 cable”) in order to avoid undue trauma to a biopsy patient, and in order to be used to detect small lesions. Generally, smaller impedance probes are able to measure smaller lesions or other tissue structures.

FIG. 1B is a cross section of the impedance probe of FIG. 1A. The body 20 of the impedance probe includes a low-loss, TEFLON®-based dielectric material (“dielectric material” 22 between the outer conductor 24 and the center conductor 26. The center conductor 26 is silver plated copper. The outer conductor 24 is tin plated on the outside only. The outer conductor 24, outer conductor end 18, and center conductor end 14 are plated with nickel (not shown) for in-vivo use. Alternatively, the center and outer conductors are fabricated from stainless steel or other biocompatible conductor that does not require plating; however, stainless steel is lossy at microwave frequencies. Therefore, plating the outer surfaces of tinned copper semi-rigid coaxial cable with nickel is particularly desirable.

A dielectric sealant 28 fills the end of the impedance probe 10 from which the dielectric material has been removed by a machining operation. The dielectric sealant 28 has a slightly higher dielectric coefficient than the dielectric material 22, and the dimensions of the center conductor and the outer conductor are adjusted to maintain a uniform transmission characteristic along the impedance probe to the end of the impedance probe. The outer conductor 24 includes a thinned outer conductor portion 24′, which increases the inner diameter of the outer conductor, and a thinned center conductor portion 26′, which decreases the outer diameter of the center conductor. The spacing (i.e. radial thickness of the dielectric sealant) between the center conductor and the outer conductor is increased where the conductors have been thinned, thus maintaining the electric impedance (e.g. 50 ohms) of the impedance probe 10. A small groove 30 is cut at end of the thinned center outer conductor portion 24′ opposite the sealed tip (see FIG. 1A, ref. num. 12) to aid in the retention of the dielectric sealant 28. The groove 30 slightly disrupts the impedance along the impedance probe, but this disruption does not significantly degrade the measurement capability of the impedance probe 10, and is easily calibrated out.

The thinned portions of the conductors and the groove 30 are formed by machining operations that can leave small metal particles embedded in the dielectric material 22. It was found that such metal particles can disrupt the impedance along the impedance probe and degrade measurement capability. One technique for removing the metal particles is to remove a thin layer of the dielectric material after the metal machining operations are complete. This mechanical removal of a small amount of dielectric material, less than about 0.1 mm deep, forms a step 32 in the dielectric sealant 28. The diameter of the step 32 is less than the optimal diameter of dielectric sealant; however, it was found that removing the small amount of dielectric material to form the step provided superior electrical performance of the impedance probe 10 over probes in which the metal particles were not removed and in which no step was formed. The total depth of dielectric sealant is about 1 mm.

STYCAST® E-1050™ epoxy encapsulant is particularly desirable for use with coaxial cable in which the center and outer conductors are copper or tin-plated copper because the thermal expansion coefficient (“TEC”) of STYCAST® E-1050™ epoxy encapsulant is closely matched (within a few parts-per-million) to that of copper. For example, copper has a TEC of about 16.5 E-6 and E-1050™ has a TEC of about 18.0 E-6, which results in a difference of about 1.5 ppm. In an alternative embodiment, a difference between the TEC of the outer conductor and the dielectric sealant is less than about 5 ppm. Thus, the end of the impedance probe remains sealed over a wide range of temperatures and after thermal cycling.

Sealed impedance probes were immersed in isopropyl alcohol at 23-25 degrees Celsius (room temperature) for four days without appreciable degradation of electrical performance. Alcohol is used to test the seal because it has relatively low surface tension and is easily taken up into the impedance probe if the end is not completely sealed. Presence of alcohol in the impedance probe is easily detected by measuring the electrical characteristics of the impedance probe with a network analyzer. If the end of the impedance probe is not sealed, fluids can leak into the probe and alter its impedance, resulting in inaccurate measurements.

A two-temperature, low-stress cure (specified by the manufacturer), was used to cure the E-1050™ epoxy encapsulant, and a vacuum was applied to remove air bubbles while the end of the impedance probe was dipped in liquid encapsulant. During the low-stress curing process the impedance probe is heated to 150 degrees Celsius, which causes thermal expansion of the conductors and of the dielectric material 22. A low-loss TEFLON®-based dielectric material contains a lot of air and has less thermal expansion than standard TEFLON®-based dielectric materials commonly used in semi-rigid coaxial cables, and the low-loss TEFLON®-based dielectric material is particularly desirable because it has less tendency to push the encapsulant out the tip of the impedance probe when the assembly is heated to cure the encapsulant. In alternative embodiments other materials and combinations of materials are used. For example, a different sealant is used with a coaxial cable made from stainless steel, which has a different thermal expansion coefficient than copper. Similarly, a sealant having a lower curing temperature is used with a copper-conductor coaxial cable and standard TEFLON®-based dielectric material.

It is desirable to nickel plate the exposed metal portions of the impedance probe for use in in-vivo systems. After the encapsulant 28 is cured, the end of the impedance probe 10 is machined to provide a flat end face. The opposite end of the body 20 of the impedance probe is pinched in a vise (not shown) to electrically couple the center conductor to the outer conductor. The impedance probe is plated with about 100 micro-inches of nickel in an electroplating (“hard nickel) process. Electrically coupling the center conductor 26 to the outer conductor 24 facilitates plating the center conductor end 14 with nickel. Alternatively, materials such as gold are used to plate impedance probes, or impedance probes are fabricated from metal(s) compatible for use in-vivo. In yet other embodiments, probes are used in applications that do not require biologically compatible plating of materials.

An example of coaxial cable suitable for embodiments of the invention is model UT31-TP-LL™ available from MICRO COAX of Pottstown, Pa. This coaxial cable is nominally 0.79 mm (0.031 inches) in outer diameter, and is relatively fragile. Impedance probes made from such coaxial cable are inserted into a rigid biopsy needle, with the tip of the biopsy needle being extended over the end of the impedance probe. The biopsy needle provides a cutting point for insertion into a patient, as well as supporting the impedance probe. In some embodiments, the biopsy needle includes one or more structures that facilitate identifying the position of the cutting point as it is inserted into the patient. When the point of the needle is at the desired location within the patient, the biopsy needle is retracted slightly to expose the end of the impedance probe to make an impedance measurement. In a particular embodiment, an 18 gauge biopsy needle is used with 0.79 mm semi-rigid coaxial cable.

FIG. 2A is a simplified cross section of biopsy probe 34 according to an embodiment of the present invention. The biopsy probe 34 includes an impedance probe 10 inside a biopsy needle 42. The biopsy needle is made from stainless steel tubing that has been sharpened to form a cutting point 44. The cutting point 44 of the biopsy needle 42 extends beyond the end 12 of the impedance probe 40. This configuration facilitates insertion into a patient because the end of the impedance probe does not interfere with the cutting action of the biopsy needle. Alternative embodiments use a biopsy needle with a different cutting point.

FIG. 2B shows the biopsy probe 34 of FIG. 2A with the biopsy needle 42 retracted to expose the end 12 of the impedance probe 10. This configuration facilitates making an impedance measurement with the impedance probe. The biopsy needle 42 is typically retracted about 2-5 mm after insertion to the desired position to expose the end of the impedance probe sufficiently that the biopsy needle does not interfere with the impedance measurement. In an embodiment, the biopsy needle is automatically retracted a selected distance after insertion. Providing the impedance probe inside of the biopsy needle advantageously allows use of a smaller diameter impedance probe compared to using a conventional impedance probe, which enables measuring a smaller volume (i.e. detect smaller tumors), can measure at a higher frequency, and causes less patient discomfort.

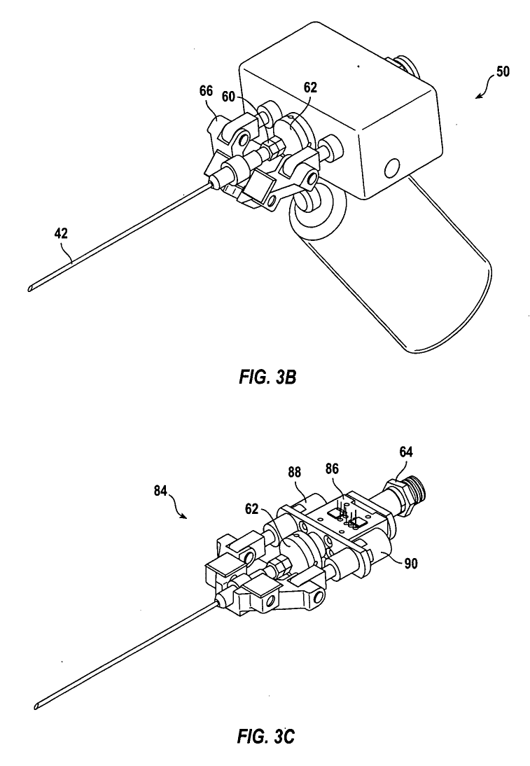

FIG. 3A is an isometric view of an automated biopsy impedance probe 50, which will be referred to as “the automated probe” for simplicity of discussion. The automated probe 50 has a handle 52 adapted to be grasped by an operator (e.g. the physician). A button 54 on the handle allows the operator to manipulate and operate the automated probe 50 with one hand. A probe head 56 includes an electronic calibration module (see FIG. 3C, ref. num. 86) and linear stepper motors (see FIG. 3C, ref. nums. 88, 90) that retract the biopsy needle 42 after it has been inserted to the desired position in the patient when the button 54 is depressed. Depressing the button (trigger) initiates a measurement sequence, such as a calibration or a biopsy test. The linear stepper motors and electronic calibration module are powered by batteries (not shown) in the handle, or alternatively is powered by a cable, such as a universal serial bus cable or separate power cable, from an external power supply. The biopsy needle 42 is a commercially available biopsy needle with a needle socket, also known as a needle connector, 58 on the end of the biopsy needle opposite the cutting point 44. Several types of needle sockets are available. Generally, the socket head is chosen according to the mating portion on the probe head 56, which is designed to mate with a particular type of needle socket, or alternatively several types of needle sockets.

The body of the impedance probe is inside the biopsy needle 42, and hence is not shown in this view. A microwave connector 60 of the impedance probe is mechanically and electrically connected to a first microwave port 62 on the probe head 56. The probe head has a second microwave port 64 with a microwave test cable interface for connection to a microwave test cable (not shown), which is connected to a vector network analyzer (“VNA”) (not shown). Those of skill in the art are familiar with the techniques of attaching microwave connectors to semi-rigid coaxial cables. Alternatively, the second microwave port 64 is omitted, and a test cable is integrated with the automated biopsy impedance probe.

A suitable VNA for use with embodiments of the invention is a PNA Model E8364B™ available from AGILENT TECHNOLOGIES, INC. of Palo Alto, Calif. It is generally desirable to use a VNA, cable, connectors, and impedance probe capable of operating at 50 GHz when looking for differences in moisture content and/or structural changes between tissues, such as between normal tissue and a cancerous lesion. Alternatively, the VNA and associated components are capable of making impedance measurements between about 100 MHz and about 20 GHz. Different frequencies are optimal for different applications. Sealed impedance probes according to embodiments of the invention enable impedance measurements to 50 GHz.

The probe head 56 has a movable carriage 66. The carriage 66 has a clamp 68 that secures the bayonet socket 58 of the biopsy needle 42. The carriage 66 rotates on hinges 70, 72 to allow removal or attachment of the biopsy needle 42 to the probe head 56 (see FIG. 3B for an illustration of the carriage 66 rotated to allow removal of the biopsy needle 42). The carriage 66 is also linearly movable to and from the probe head 56. Linear stepper motors (see FIG. 3C, ref. nums. 88, 90) in the probe head 56 are supported by bushings 78, 80. The linear stepper motors are used to automatically retract the carriage (which is attached to the needle), thus exposing the tip of the impedance probe. When the button 54 is depressed, the probe head automatically retracts the carriage 66, and hence the biopsy needle 42, a selected amount to expose the end of the impedance probe, which is removably attached to the first microwave port 62 of the probe head. The impedance probe and biopsy needle are removably attached to the probe head to allow convenient replacement of these components between biopsies. Alternatively, only the biopsy needle is replaced between procedures, or is removed to allow calibration or re-calibration of the impedance probe. In some applications, ethylene oxide (“EtO”) is used to sterilize impedance probes.

FIG. 3B is an isometric view of the automated probe 50 with the carriage 66 rotated down to allow removal of the biopsy needle 42. The microwave connector 60 is unscrewed from the first microwave port 62 to remove the impedance probe, if desired. The biopsy needle can be removed without removing the impedance probe.

FIG. 3C is an isometric view of a portion 84 of the automated probe of FIGS. 3A and 3B. An electronic calibration module (“E-cal module”) 86 is in the microwave path between the first microwave port 62, which is connected to the impedance probe, and the second microwave port 63, which in use is connected to a test cable (not shown), which is connected to a VNA (not shown) or other electronic test instrument. The E-cal module 86 is similar to the electronic portion of the E-cal module number N4694A™ available from AGILENT TECHNOLOGIES, INC. of Palo Alto, Calif. The E-cal module 86 is controlled by the VNA over a link, such as a universal serial bus (“USB”) cable or other interface.

The linear stepper motors 88, 90 are actuated by depressing the button 54 (see FIG. 3A) on the handle of the automated probe. Speed of operation and distance of travel is selected prior to performing a biopsy and the linear stepper motors are controlled by a controller incorporated in the automated probe, or by an external controller.

An accurate calibration of a microwave measurement system (for example, the VNA, cables, connectors, and probe) is important to obtain accurate impedance measurements. When microwave devices are tested in a microwave measurement system, a calibration is usually done at the test ports (i.e. the ports at which the devices under test will be attached to for measurement) of the measurement system by attaching various calibration standards and measuring the resultant network parameters. One example of this type of calibration uses a set of short-load-open-termination (“SLOT”) calibration standards. However, in microwave component test systems, the test cables usually do not move significantly between the calibration and device testing. When the automated probe is used in a biopsy, the cables move in order to allow the biopsy needle to be inserted into the patient.

The E-cal module 86 incorporated into the automated probe allows calibrating out measurement errors arising from cable movement, or alternatively indicating that the measurement errors were too great to be calibrated out and providing an indication, such as a distinctive beeping tone, that the measurement is inaccurate. In a particular embodiment, one beeping tone indicates that a successful biopsy impedance measurement was completed, and a different beeping tone indicates that a biopsy impedance measurement was unsuccessful (i.e. potentially inaccurate). Thus, the physician operating the probe knows whether the biopsy impedance measurement was valid, or needs to be repeated because the cable was moved during the measurement, for example.

The impedance probe is calibrated at its end before beginning a biopsy impedance measurement. An open calibration is done by extending the end of the impedance probe past the cutting tip of the biopsy needle. Alternatively, a calibration is done with the biopsy needle removed from the automated probe. The automated probe is connected to the VNA with the test cable that will be used during the biopsy impedance measurement. A short calibration is done by pressing the end of the impedance probe against an elastomeric conductor to short the center conductor end to the outer conductor end of the impedance probe together. A load calibration is done by immersing the end of the impedance probe into a liquid, such as water or isopropyl alcohol. It is not necessary that the liquid provide the characteristic impedance of the impedance probe. The load provided by liquid is characterized and sufficiently stable to result in a suitable calibration of the impedance probe in a biopsy measurement. An E-cal is done and the calibration values are stored in the VNA. The calibration data is stored in the E-cal module so that subsequent measurements can be corrected for cable movement.

The E-cal module 86 enables quick, automatic re-calibration at the plane of the second microwave port 64, and thus can calibrate errors arising from movement of the test cable. The E-cal module 86 does not calibrate out errors arising from flexing of the impedance probe, but the biopsy needle rigidly supports the impedance probe, and the automatic linear motion of the biopsy needle relative to the impedance probe minimizes flexing of the impedance probe.

In one embodiment, a method of taking a biopsy includes, calibrating an impedance probe. In a particular embodiment, the impedance probe and biopsy needle are connected to a carriage of an automated biopsy impedance probe (see, e.g., FIG. 3A), which is connected to a VNA, and the biopsy needle is sufficiently retracted to expose the tip of the impedance probe for calibration. In a particular embodiment, the calibration includes open, short, and load measurements. The impedance probe is slidably disposed within the biopsy needle so as to cover the tip of the impedance probe (ref. FIG. 2A) and the biopsy needle is inserted into a subject so that the point of the biopsy needle is near a desired biopsy location. The biopsy needle is then sufficiently retracted to expose the tip of the impedance probe for an impedance measurement within the selected tissue or fluid. A calibrated biopsy impedance measurement of the selected tissue or fluid is taken by a VNA, and the measured values are stored.

In a particular embodiment, retraction and measurement steps are part of an automated sequence initiated by depressing a button on the automated biopsy probe. In a particular embodiment, the automated biopsy probe includes an E-cal module that compensates for cable movement. In a further embodiment, a first audible sound is emitted by the automated probe, the VNA, or another device, if the biopsy impedance measurement is valid (i.e., within the E-cal correction limits), and emits a different audible sound if the biopsy measurement is not valid. In one embodiment, depressing the button a second time initiates another biopsy impedance measurement. Alternatively, depressing the button a second time extends the biopsy needle to cover the tip of the impedance probe.

While the preferred embodiments of the present invention have been illustrated in detail, it should be apparent that modifications and adaptations to these embodiments might occur to one skilled in the art without departing from the scope of the present invention as set forth in the following claims.

What is claimed is:

1. A biopsy probe comprising:
a biopsy needle, and
an impedance probe slidably disposed within the biopsy needle.

2. The biopsy probe of claim 1 wherein the impedance probe is a coaxial cable having a center conductor end and an outer conductor end comprising a biocompatible metal.

3. The biopsy probe of claim 1 wherein the impedance probe is a coaxial cable having a center conductor end and an outer conductor end plated with nickel.

4. The biopsy probe of claim 1 wherein the impedance probe has a low-loss dielectric material disposed between a metal outer conductor and a metal center conductor.

5. The biopsy probe of claim 1 wherein the impedance probe has
a thinned outer conductor portion;
a thinned center conductor portion; and
a dielectric sealant disposed between the thinned outer conductor portion and the thinned center conductor portion of the impedance probe.

6. The biopsy probe of claim 5 wherein the dielectric sealant has a first thermal expansion coefficient and the thinned outer conductor portion has a second thermal expansion coefficient, a difference between the first thermal expansion coefficient and the second thermal expansion coefficient being less than 5 ppm.

7. The biopsy probe of claim 5 wherein the dielectric sealant comprises epoxy.

8. The biopsy probe of claim 5 further comprising a groove in the thinned outer conductor portion filled with the dielectric sealant.

9. The biopsy probe of claim 1 wherein an outer diameter of the impedance probe is not greater than 0.79 mm.

10. The biopsy probe of claim 9 wherein the biopsy needle is not larger than an 18 gauge needle.

11. The biopsy probe of claim 1 further comprising:
a handle;
a probe head;
a carriage movably coupled to the probe head having a clamp configured to removably secure the biopsy needle to the carriage; and
a first microwave port configured to removably secure the impedance probe to the probe head.

12. The biopsy probe of claim 11 further comprising a second microwave port having a microwave test cable interface.

13. The biopsy probe of claim 11 further comprising an electronic calibration module in the automated biopsy impedance probe.

14. The biopsy probe of claim 11 further comprising an electronic motor coupled to the carriage and to the probe head capable of extending and retracting the carriage with respect to the probe head.

15. The biopsy probe of claim 11 wherein the electric motor is a linear stepper motor configured to automatically retract the carriage a selected distance so as to expose a selected amount of the impedance probe extending from the biopsy needle.

16. The biopsy probe of claim 15 wherein a tip of the impedance probe extends about 2 mm to about 5 mm beyond a cutting point of the biopsy needle when the carriage is retracted.

17. The biopsy probe of claim 15 wherein the linear stepper motor is configured to automatically extend the biopsy needle from a retracted position so as to cover a tip of the impedance probe.
